(19) United States
(12) Patent Application Publication
(10) Pub. No.: US 2013/0115661 A1
(43) Pub. Date: May 9, 2013

(54) METHOD FOR PRODUCING BIOBASED CHEMICALS FROM CULTIVATED PLANT BIOMASS

(71) Applicants: John R. Peterson, Chardon, OH (US); Christopher M. Yost, Ayr (CA); Jian Wu, Guelph (CA)

(72) Inventors: John R. Peterson, Chardon, OH (US); Christopher M. Yost, Ayr (CA); Jian Wu, Guelph (CA)

(73) Assignee: THESIS CHEMISTRY, LLC, Mentor, OH (US)

(21) Appl. No.: 13/292,632

(22) Filed: Nov. 9, 2011

(51) Int. Cl. C12P 7/02; C07D 307/02; C07D 315/00; C12P 7/22; C12P 7/40; C12P 5/00; C12P 5/02; C12P 7/24; C12P 7/26; C12P 7/62; C12P 19/04; C08B 1/00; C07C 51/42; C07C 67/60; C12P 7/06; C07C 29/88; C07C 45/85; C07C 1/20; C07C 11/18; C12P 17/04

(52) U.S. Cl. 435/101; 435/126; 549/429; 549/263; 435/156; 435/136; 435/155; 435/166; 435/167; 435/147; 435/148; 435/135; 536/56; 562/513; 560/129; 562/405; 560/8; 435/161; 568/840; 568/420; 568/303; 568/852; 585/469; 585/600

(57) ABSTRACT

A method for utilizing cultivated plant biomass components, namely cellulose, hemicellose, and lignin, and converting them to value-added biobased chemical products is described herein. The present method provides treatments to obtain a plurality of component streams from cultivated plant biomass for producing derivative products while minimizing waste products.

### I. BACKGROUND OF THE INVENTION

A. Field of Invention

The present invention is directed generally to a method of the production of value-added, biobased chemical products from a plurality of component streams from cultivated plant biomass. The present method further provides treatments to obtain the plurality of component streams from cultivated plant biomass.

B. Description of the Related Art

The world currently faces depletion of fossil fuels while demands for these fuels are ever increasing. Petrochemicals provide an energy source and a component of the majority of raw materials used in many industries. In fact, approximately 95% of all chemicals manufactured today are derived from petroleum. However, this heavy reliance upon fossil fuels is creating harm to the environment. The burning of these fossil fuels has led to the pollution of air, water and land, as well as global warming and climate changes. Through the use of fossil fuels, the environment has been harmed, perhaps irreparably, in an effort to meet the nearly insatiable demand for energy and manufactured products. Fossil fuels are a finite natural resource, with the depletion of readily available oil reserves across the globe; the supply chain has shifted to more complex and environmentally risky production technologies. A reduction in use and conservation of fossil fuels is clearly needed. Some alternatives to fossil fuels, like solar power, wind power, geothermal power, hydropower, and nuclear power, are used to a degree. However, a more efficient use of renewable resources is always being sought.

As a stable and independent alternative to fossil fuels, cultivated plant biomass has emerged as a potentially inexhaustible resource for the production of energy, transportation fuels, and chemicals. The advantage in turning to domestic, renewable cultivated plant biomass for such purposes would be magnified during periods of an oil crisis, a price surge, or political unrest within oil producing regions of the world. Cultivated plant biomass can be employed as a sustainable source of energy and is a valuable alternative to fossil fuels in the production of chemicals. More specifically, the biorefining of cultivated plant biomass into derivative products typically produced from petroleum could help to lessen the dependence on foreign crude oil. Cultivated plant biomass can become a key resource for chemical production in much of the world. Moreover, cultivated plant biomass, unlike petroleum, is renewable. Cultivated plant biomass can provide sustainable substitutes for petrochemically derived feedstocks used in existing markets.

Cultivated plant biomass can be comprised of plants whose origin or selection is primarily due to intentional human activity. Cultivated plant biomass can be made up primarily of cellulose, hemicellulose, and lignin. These components, if economically separated from one another, can provide vital sources of chemicals normally derived from petrochemicals. The use of cultivated plant biomass can also be beneficial with cultivated plants that are sparsely used and by-products, residues and wastes that currently have little or no use. Cultivated plant biomass can provide valuable chemicals and reduce dependence on coal, gas, and fossil fuels, in addition to boosting local and worldwide economies.

The use of cultivated plant biomass in the production of chemicals historically has focused mostly on bioethanol. Cellulosic bioethanol production requires a breakdown of the cultivated plant biomass into component streams with often only the cellulose component utilized. The OrganoSolv™ and Alcell® processes can be used to efficiently separate cellulose from cultivated plant biomass under mild conditions, namely through the use of an aqueous organic solvent, usually ethanol. These processes provide the simultaneous removal of the hemicellulose sugar and lignin in separated streams. Even though an organic solvent is used during this process, it can be recycled and used again in the process. Alternatively, separate component streams can be obtained from cultivated plant biomass through at least one of kraft pulping, sulfite pulping, steam explosion, ammonia fiber explosion, dilute acid hydrolysis, alkaline hydrolysis, alkaline oxidative treatment, enzymatic hydrolysis, pyrolytic processes, and enzymatic treatment.

Although cultivated plant biomass has garnered attention as a feedstock for bioethanol and solid biofuel, the intrinsic value of the components of cultivated plant biomass in chemical production continues to be largely overlooked. Other than fossil fuels, lignin is the most abundant source of aromatic chemicals. Lignin can be used in developing technologies that transform cultivated plant biomass into value-added, aromatic chemicals. In addition, the hemicellulose portion of cultivated plant biomass can also be converted into useful biobased chemicals.

The present invention provides methods for producing a plurality of component streams from cultivated plant biomass, namely cellulose, hemicellose, and lignin, and converting these component streams into value-added biobased chemicals while minimizing waste products.

### II. SUMMARY OF THE INVENTION

Accordingly, it is an object of the present invention to provide a method for biorefining. It may include the steps of providing cultivated plant biomass and treating said cultivated plant biomass to provide a plurality of component streams. The method may further include producing derivative products from the plurality of component streams.

One object of the present invention includes cultivated plant biomass that comprises cultivated plants, cultivated trees, cultivated shrubs, and cultivated bushes.

Yet another object of the present invention includes cultivated plant biomass that comprises at least one of aquatic plants, medicinal plants, fiber plants, ornamental plants, and grassy plants.

Still another object of the present invention includes cultivated plant biomass that comprises at least one of annual cultivated plants, native annual cultivated plants, hybrid annual cultivated plants, and genetically modified annual cultivated plants.

Still yet another object of the present invention includes cultivated plant biomass that comprises at least one of perennial cultivated plants, native perennial cultivated plants, hybrid perennial cultivated plants, and genetically modified perennial cultivated plants.

Yet another object of the present invention includes cultivated plant biomass that comprises at least one of biennial cultivated plants, native biennial cultivated plants, hybrid biennial cultivated plants, and genetically modified biennial cultivated plants.

A further object of the present invention includes cultivated plant biomass that comprises cultivated plant residues of at least one of cultivated plants, cultivated trees, cultivated shrubs, cultivated bushes, aquatic plants, medicinal plants, fiber plants, ornamental plants, grassy plants, annual cultivated plants, native annual cultivated plants, hybrid annual cultivated plants, genetically modified annual cultivated plants, perennial cultivated plants, native perennial cultivated plants, hybrid perennial cultivated plants, genetically modified perennial cultivated plants, biennial cultivated plants, native biennial cultivated plants, hybrid biennial cultivated plants, and genetically modified biennial cultivated plants.

Yet another object of the present invention is processing cultivated plant biomass to provide a plurality of component streams.

Still another object of the present invention is the plurality of component streams comprises lignin, cellulose, and hemicellulose.

Another object of the present invention is treating cultivated plant biomass by mechanical processing and component separation processing.

According to one embodiment of the present invention, mechanical processing comprises at least one of chopping, chipping, cutting, shredding, debarking, milling, and grinding.

Yet another object of the present invention is treatment of cultivated plant biomass by component separation processing provides cellulose.

In another embodiment of the present invention, component separation processing provides a mixture of hemicellulose and lignin.

Still another object of the present invention is treating cultivated plant biomass by component separation processing provides lignin and hemicellulose as separated components.

Still yet another object of the present invention is to treat cultivated plant biomass by optional chemical processing.

According to one embodiment of the present invention, optional chemical processing comprises at least one of solvent treatment, acidic treatment, basic treatment, or enzymatic treatment.

According to another embodiment of the present invention, extractables are separated from the cultivated plant biomass by optional chemical processing.

According to yet another embodiment of the present invention, the extractables removed can be used for at least one of producing biofuels, lubricating, cleaning, disinfecting, deodorizing, scenting, and metal extracting from ores.

Another object of the present invention is the production of derivative products from the plurality of component streams.

In another embodiment of the present invention, chemicals used in optional chemical processing are recovered and recycled.

According to another embodiment of the present invention, component separation processing includes a residual chemical removal with optional pH adjustment.

Another embodiment of the present invention provides hemicellulose and lignin as part of residual chemical removal.

According to one embodiment of the invention, a chemical can be recovered and recycled from the residual chemical removal.

Yet another object of the present invention is an additional treatment comprising of at least one of heat treatment, pressure treatment, kraft pulping, sulfite pulping, pyrolysis, steam explosion, ammonia fiber explosion, dilute acid hydrolysis, alkaline hydrolysis, alkaline oxidative treatment, and enzymatic treatment.

Another embodiment of the present invention includes removing extractables from the additional treatment of the component separation processing.

Yet another embodiment of the present invention includes recovering and recycling at least one chemical from the additional treatment.

According to one embodiment of the invention, one of the component streams is selectively utilized.

According to another embodiment of the invention, at least two of the component streams are selectively utilized.

According to one embodiment of the invention, the plurality of component streams is a mixture of the plurality of component streams.

According to still yet another embodiment of the invention, an independent component stream is a mixture of the plurality of component streams.

According to still another embodiment of the invention, an independent component stream is independent and separate from the plurality of component streams.

Yet another object of the present invention is the production of one of more derivative products from an independent and separate component stream.

Another object of the present invention according to one embodiment of the invention is utilizing at least one of the component streams for producing the derivative products.

Still yet another object of the present invention is use of the residue component stream from production of at least one derivative product in the production of other biobased chemicals.

Still another object of the present invention is at least one of the derivative products comprises commodity chemicals, fine chemicals, and specialty chemicals.

Yet another object of the present invention is producing at least one derivative product comprises at least one chemical process, biological process, catalytic process, and pyrolytic process.

According to one object of the present invention, the derivative products of lignin comprise at least one of aromatic chemicals and fuels.

According to one embodiment of the present invention, the derivative products of lignin comprise at least one of aromatic carboxylic acids, aromatic esters, aromatic aldehydes, aryl alcohols, aryl ketones, styrenes, aryl ethanes, aryl propenes, aryl propanes, cresols, phenols, benzenes, and pyrolytic oils.

According to another embodiment of the present invention, the derivative products of lignin may comprise but are not limited to methyl and ethyl 4-hydroxybenzoate, methyl and ethyl vanillate, methyl and ethyl syringate, 4-hydroxybenzoic acid, (4-hydroxyphenyl)acetic acid, vanillic acid, homovanillic acid, syringic acid, homosyringic acid, 4-hydroxybenzaldehyde, vanillin, syringaldehyde, 4-hydroxybenzyl alcohol, 2-(4-hydroxyphenyl)ethanol, vanillyl alcohol, homovanillyl alcohol, syringyl alcohol, homosyringyl alcohol, 4-hydroxyacetophenone, acetoguaiacone, acetosyringone, 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, iso-syringeugenol, ethyl phenol, ethyl guaiacol, ethyl syringol, propyl phenol, propyl guaiacol, propyl syringol, cresol, creosol, syringyl creosol, phenol, guaiacol, syringol, benzene, toluene, xylene, ethyl benzene, propyl benzene, biphenyl, and pyrolytic oils.

Another object of the present invention is the derivative products of cellulose comprise at least one of aliphatic chemicals, heterocyclic chemicals, and fuels.

According to one embodiment of the present invention, the derivative products of cellulose comprise at least one of cellulosic esters, aliphatic carboxylic acids, aliphatic esters, polyols, furans, dihydrofurans, tetrahydrofurans, lactones, and ethanol.

According to another embodiment of the present invention, the derivative products of cellulose may comprise but are not limited to cellulose acetate, cellulose propionate, cellulose benzoate, methyl and ethyl adipate, methyl and ethyl levulinate, methyl and ethyl succinate, methyl and ethyl 2,5-furandicarboxylate, adipic acid, levulinic acid, succinic acid, 2,5-furandicarboxylic acid, 3,4-dehydro-γ-valerolactone, γ-valerolactone, 2-methyltetrahydrofuran, sorbitol, hexane-1,6-diol, pentane-1,4-diol, butane-1,4-diol, 2,5-di(hydroxymethyl)furan, 2,5-di(hydroxymethyl)tetrahydrofuran, glyercol, propylene glycol, and ethanol.

Yet another object of the present invention is the derivative products of hemicellulose comprise at least one of aliphatic chemicals, heterocyclic chemicals, and fuels.

According to one embodiment of the present invention, the derivative products of hemicellulose comprise at least one of polyols, furans, dihydrofurans, tetrahydrofurans, lactones, and butenes.

According to another embodiment of the present invention, the derivative products of hemicellulose may comprise but are not limited to furfural, γ-butyrolactone, tetrahydrofuran, ribitol, arabitol, xylitol, glyercol, propylene glycol, and isoprene.

Still another object of the present invention is that the chemicals used for processing the cultivated plant biomass are recoverable for reuse.

Still yet another object of the present invention is that the plurality of derivative products comprises at least one of achiral, racemic, and optically pure products.

Still another object of the present invention is that at least one derivative product can be used in the production of other chemicals, materials, and products.

Yet another object of the present invention according to one embodiment of the invention is that the cultivated plant biomass has a weight, and a waste product of the cultivated plant biomass is less than 25% of the cultivated plant biomass weight.

Still yet another object of the present invention according to one embodiment of the invention is that the cultivated plant biomass has a weight, and a waste product of the cultivated plant biomass is less than 15% of the cultivated plant biomass weight.

Further, another object of the present invention is to provide a method for producing energy utilizing the waste product of the cultivated plant biomass.

According to another aspect, the present invention provides a method for biorefining which may comprise the steps of providing cultivated plant biomass, processing the cultivated plant biomass to provide a plurality of component streams, and using waste product from the plurality of component streams to produce energy.

According to another aspect of the present invention, the energy is heat or power.

Another object of the present invention is that it provides a method for biorefining which may comprise the steps of providing cultivated plant biomass, processing the cultivated plant biomass to provide a plurality of component streams by mechanical processing, component separation processing, optional chemical processing, residual chemical removal, and an additional treatment, providing a plurality of component streams comprising lignin, cellulose, and hemicellulose from the cultivated plant biomass, recovering chemicals used for chemical processing and residual chemical removal for reuse, removing extractables, reducing the waste product of the cultivated plant biomass, and producing at least one of aromatic carboxylic acids, aromatic esters, aromatic aldehydes, aryl alcohols, aryl ketones, styrenes, aryl ethanes, aryl propenes, aryl propanes, cresols, phenols, benzenes, and pyrolytic oils, cellulosic esters, aliphatic carboxylic acids, aliphatic esters, polyols, furans, dihydrofurans, tetrahydrofurans, lactones, ethanol, and butenes, from at least one of the component streams.

Another object of the present invention is to provide a method for biorefining that is cost effective.

Further, another object of the present invention is to provide a method for biorefining that is easy to implement and use.

Still other benefits and advantages of the present invention will become apparent to those skilled in the art to which it pertains upon a reading and understanding of the following detailed specification.

### III. BRIEF DESCRIPTION OF THE DRAWINGS

The invention may take physical form in certain parts and arrangement of parts, embodiments of which will be described in detail in this specification and illustrated in the accompanying drawings which form a part hereof, and wherein:

FIG. 1 is a flow diagram schematically illustrating the present invention.

FIG. 2 is a flow diagram schematically illustrating another aspect of the present invention.

FIG. 3 is a flow diagram schematically illustrating another aspect of the present invention.

FIG. 4 is a flow diagram schematically illustrating another aspect of the present invention.

FIG. 5 is a flow diagram schematically illustrating another aspect of the present invention.

FIG. 6 is a flow diagram schematically illustrating another aspect of the present invention.

FIG. 7 is a flow diagram schematically illustrating another aspect of the present invention.

### IV. DETAILED DESCRIPTION OF THE INVENTION

Referring now to the drawings wherein the showings are for purposes of illustrating embodiments of the invention only and not for purposes of limiting the same. Relative language used herein is best understood with reference to the drawings, in which like numerals are used to identify like or similar items.

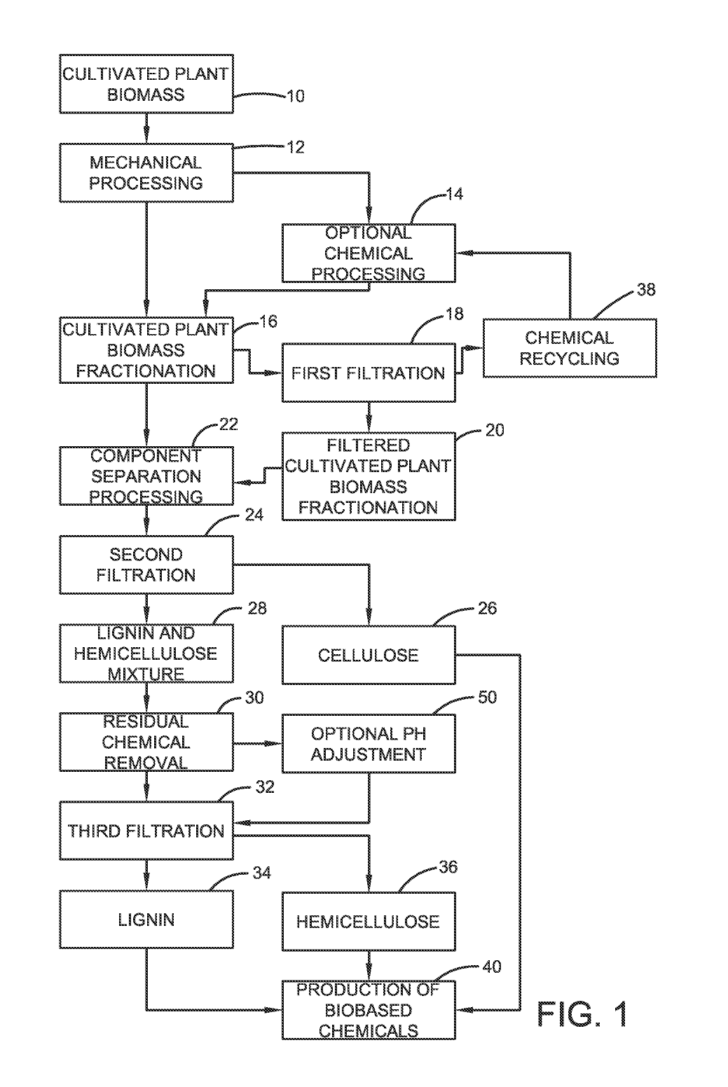

FIG. 1 shows a flow diagram schematically depicting the general overview for the illustrative flow for treating and processing cultivated plant biomass 10 for the production of biobased chemicals 40 in accordance with an embodiment of the present invention. First, cultivated plant biomass 10 may be obtained for processing. Cultivated plant biomass 10 can be comprised of plants whose origin or selection is primarily due to intentional human activity. Some examples of cultivated plant biomass 10 can include but are not limited to cultivated plants like switchgrass, miscanthus, hemp, King grass, sugarcane, sweet sorghum, duckweed, and a variety of cultivated tree species ranging from eucalyptus to palm oil. Some of this cultivated plant biomass 10 may be grown for the production of fuels and chemicals. Because of their typical higher biomass density per acre, cultivated plant biomass 10 may provide a biomass source where they are utilized, particularly in geographies and/or soils where certain higher value agricultural and woody biomass do not grow as well. In other words, cultivated plant biomass 10 may provide a use for marginal lands where environmental stress is inherent and the land cannot be used for typical agricultural or timber purposes. In the process described herein, this cultivated plant biomass 10 may be used for the production of biobased chemicals 40. Cultivated plant biomass 10 can be received in any number of forms, including loose, bailed, wrapped, pellets, cubes, and briquettes. The cultivated plant biomass 10 may be considered to include cultivated plants, cultivated trees, cultivated shrubs, and cultivated bushes. Sources of cultivated plant biomass 10 can encompass native plants, hybrid plants, and genetically modified plants. In addition, cultivated plant biomass 10 may include annual cultivated plants, native annual cultivated plants, hybrid annual cultivated plants, genetically modified annual cultivated plants, perennial cultivated plants, native perennial cultivated plants, hybrid perennial cultivated plants, genetically modified perennial cultivated plants, biennial cultivated plants, native biennial cultivated plants, hybrid biennial cultivated plants, and genetically modified biennial cultivated plants. Cultivated plant biomass 10 may also include residential and/or commercial landscaping plants. Cultivated plant biomass 10 may include some agricultural plants, medicinal plants, fiber plants, ornamental plants, aquatic plants, and grassy plants. The cultivated plant biomass 10 described herein can also be a by-product, residue or waste product of biomass, including cultivated plant biomass 10 residues from other industries like cellulosic bioethanol refineries and pulp and paper mills. The cultivated plant biomass 10 described herein can be a by-product, residue or waste product of cultivated plants, cultivated trees, cultivated shrubs, cultivated bushes, aquatic plants, medicinal plants, fiber plants, ornamental plants, grassy plants, annual cultivated plants, native annual cultivated plants, hybrid annual cultivated plants, genetically modified annual cultivated plants, perennial cultivated plants, native perennial cultivated plants, hybrid perennial cultivated plants, genetically modified perennial cultivated plants, biennial cultivated plants, native biennial cultivated plants, hybrid biennial cultivated plants, and genetically modified biennial cultivated plants.

In essence, cultivated plant biomass 10 may include any cultivated plant source that can be added to the process to create at least one component stream, typically lignin 34, cellulose 26, and/or hemicellulose 36, for the production of biobased chemicals 40. Depending on the type of cultivated plant biomass material, the amounts of the cultivated plant biomass, and the compositions of the cultivated plant biomass, these component streams can differ.

Next, the cultivated plant biomass 10 may undergo mechanical processing 12 in order to reduce the size of the cultivated plant biomass 10 and prepare it for further processing. For the mechanical processing 12, the cultivated plant biomass can undergo chopping, chipping, cutting, shredding, debarking, milling, and grinding. In order to break down the cultivated plant biomass, there can be one or more mechanical processing 12 steps needed. The type of mechanical processing 12 may be dependent upon the type of cultivated plant biomass and its requirements for breaking it down for further treatment.

After the mechanical processing 12, the cultivated plant biomass 10 may be subjected to an optional chemical processing 14. This optional chemical processing 14 may serve to further break down the cultivated plant biomass 10 as well as remove fats, oils, resins, pitches, waxes, and other extractables. After both mechanical processing 12 and optional chemical processing 14, the cultivated plant biomass fractionation 16 can be formed.

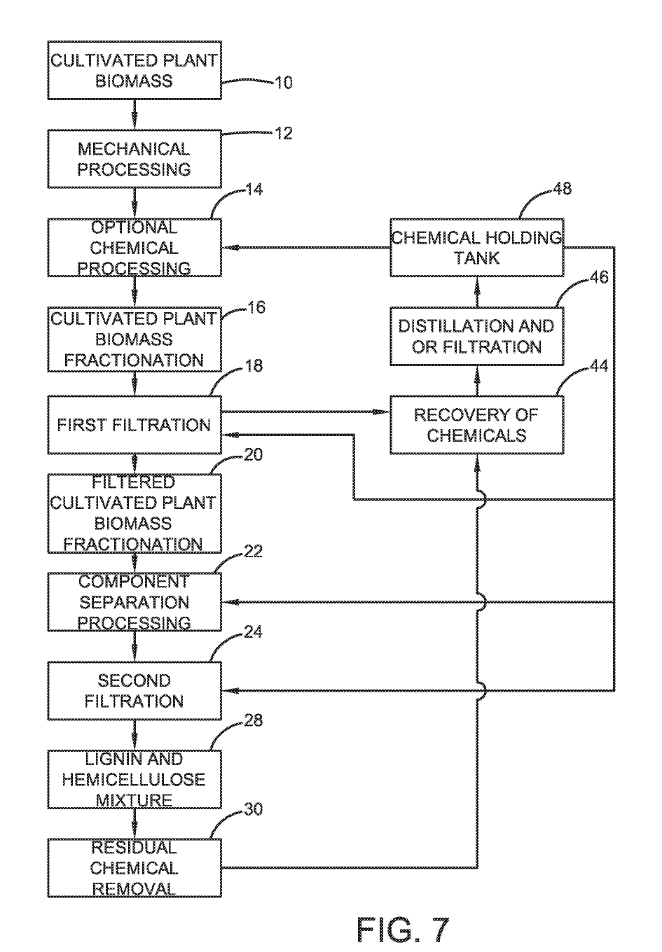

Still referring to FIG. 1, the cultivated plant biomass fractionation 16 may undergo a first filtration 18 if the optional chemical processing 14 is completed. This first filtration 18 serves to remove the optional chemical processing 14 from the cultivated plant biomass fractionation 16. The chemical processing step 14 may include fats, oils, resins, pitches, waxes, and other extractables removed in the optional chemical processing 14 which can be further separated and marketed as useful products of commerce. From the first filtration 18, the chemical used from the optional chemical processing 14 can be recycled under a chemical recycling 38 step. This chemical recycling 38 process will be detailed further in FIG. 7.

Using the cultivated plant biomass fractionation 16 can provide a greener process by utilizing at least three of the component streams of cultivated plant biomass 10. These three component streams of cultivated plant biomass 10 may include cellulose 26, hemicellulose 36, and lignin 34. Typically for cultivated plant biomass 10, the amounts of cellulose 26, hemicellulose 36, and lignin 34 will vary for different species of cultivated plant biomass 10. Depending on the type of cultivated plant biomass 10 used or targeted, the amounts of each of the component streams can determine the end products in the production of biobased chemicals 40.

After the first filtration 18 with the optional chemical processing 14, the filtered cultivated plant biomass fractionation 20 may be formed. Either the filtered cultivated plant biomass fractionation 20 from the optional chemical processing 14 or the cultivated plant biomass fractionation 16 from the mechanical processing 12 alone can be broken down even further by the component separation processing 22. In the component separation processing 22, a high pressure and temperature can successfully break down the cultivated plant biomass even further. Alternatively, the filtered cultivated plant biomass fractionation 20, or the cultivated plant biomass fractionation 16 from the mechanical processing 12 alone, can be broken down with other processes in the component separation processing 22 that may include at least one of kraft pulping, sulfite pulping, pyrolysis, steam explosion, ammonia fiber explosion, dilute acid hydrolysis, alkaline hydrolysis, alkaline oxidative treatment, and enzymatic treatment. A second filtration 24 can then be done to separate the cellulose 26 from the lignin and hemicellulose mixture 28. This lignin and hemicellulose mixture 28 can then go through both a residual chemical removal 30 and a third filtration 32 in order to separate the lignin and hemicellulose mixture 28 into lignin 34 and hemicellulose 36. Further, an optional pH adjustment 50 may take place prior to the third filtration 32 to effect a more complete separation of lignin 34 and hemicellulose 36.

With the separated component streams for cellulose 26, lignin 34, and hemicellulose 36, a production of biobased chemicals 40 can be achieved.

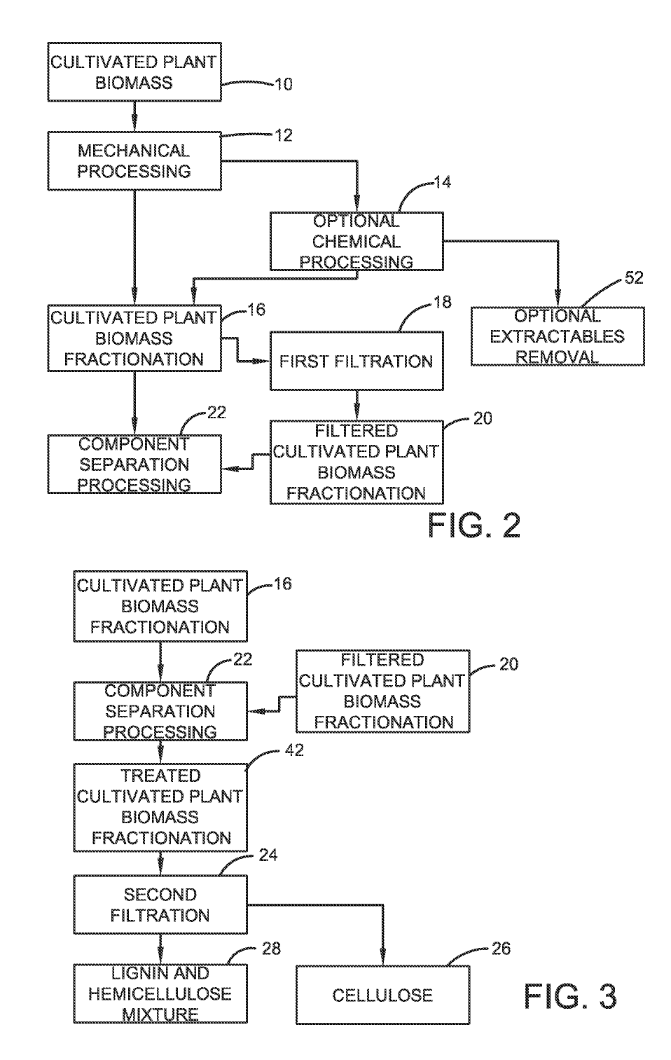

FIG. 2 is a flow diagram schematically depicting the process in which cultivated plant biomass 10 may be mechanically and optionally chemically processed to provide both a fractionated and filtered cultivated plant biomass product in accordance with an embodiment of the present invention.

In FIG. 2, the cultivated plant biomass 10 may undergo mechanical processing 12 in order to reduce the size of the cultivated plant biomass 10 and prepare it for further processing. For the mechanical processing 12, the cultivated plant biomass 10 can be delivered for processing. Depending on the type of cultivated plant biomass 10, the mechanical processing 12 can vary. The mechanical processing 12 can include chopping, chipping, cutting, shredding, debarking, milling, and grinding. No matter what type of cultivated plant biomass 10 may be used, milling or chopping may be needed in order to reduce size of the material for ease and efficiency of processing. The cultivated plant biomass 10 can be milled to various sizes, but the size of the milled cultivated plant biomass is tied to the efficiency of how it is broken down within the subsequent processes. For instance, larger particle sizes of milled cultivated plant biomass may take longer to be broken down in both the optional chemical processing 14 and later processes within the component separation processing 22 due to less surface area in which to react during the breakdown processes. Cultivated plant biomass 10 can typically be milled to a particle diameter of less than ⅛″. The maximum particle diameter for milling of cultivated plant biomass 10 can typically be about ¼″. Preferably, a uniform particle size can be reached for ease and consistency of processing during the subsequent processes.

After the mechanical processing 12, the cultivated plant biomass 10 may be subjected to an optional chemical processing 14. Cultivated plant biomass 10 may undergo the optional chemical processing 14 if additional breakdown of the cultivated plant biomass is needed. The optional chemical processing 14 of some cultivated plant biomass, like conifers, may be beneficial for production of biobased chemicals 40 of FIG. 1. This optional chemical processing 14 may typically be done through a solvent treatment. During the optional chemical process 14, the cultivated plant biomass 10 can be further broken down after the mechanical processing 12. Typically, the optional chemical processing 14 can be performed in a solvent like ethanol. Besides ethanol, other organic solvents, acids, bases, or enzymes can be used for the optional chemical processing 14. However, the use of these acids, bases, or enzymes may lead to varying degrees of hydrolysis.

This optional chemical processing 14 can also undergo an optional extractable removal 50. This optional extractable removal 50 helps to remove any extractables from the cultivated plant biomass 10. Some of these extractables can include fats, oils, resins, pitches, and waxes present in different forms of cultivated plant biomass 10. Depending on the cultivated plant biomass 10 source, the type and amount of these extractables can vary. The extractables do not have to be taken out, but their removal may allow for a purer end product with the production of biobased chemicals 40 in FIG. 1. Further, the optional extractable removal 50 may provide products of importance to commerce and for more of a comprehensive utilization of the cultivated plant biomass resource and generation of less waste. The extractables removed during the optional extractable removal 50 can be further separated, processed, and marketed as useful products of commerce for at least one of biofuels, lubricants, cleaning agents, disinfectants, deodorant additives, scents, and extraction of metal from ores.

After both mechanical processing 12 and optional chemical processing 14, the cultivated plant biomass fractionation 16 may be formed. The cultivated plant biomass fractionation 16 can then filtered to form the filtered cultivated plant biomass fractionation 20 if it was subjected to optional chemical processing 14. In this filtration after the mechanical and chemical processing, which is referred to as the first filtration 18, the optional chemical processing 14 can be partially removed from the cultivated plant biomass fractionation 16. For the first filtration 18, there are a series of steps where the cultivated plant biomass fractionation 16 may be filtered, then washed with additional chemical which is used in the optional chemical processing 14, typically ethanol or another alcohol, and then filtered again to remove some of the chemical from the optional chemical processing 14. After this wash and first filtration 18, typically about 50% of the chemical may be typically removed. The filtered cultivated plant biomass fractionation 20 may or may not contain some of the chemical from the optional chemical processing 14 step. From this step, either the filtered cultivated plant biomass fractionation 20 or the cultivated plant biomass fractionation 16 will be subjected to component separation processing 22 as detailed in FIG. 3.

With reference now to FIG. 3, the flow diagram schematically depicts the process in which the cultivated plant biomass fractionation 16 or the filtered cultivated plant biomass fractionation 20 may be processed further to obtain a treated cultivated plant biomass fractionation 42 in accordance with an embodiment of the present invention. The cultivated plant biomass fractionation 16 or the filtered cultivated plant biomass fractionation 20 can be subjected to a component separation processing 22. This component separation processing 22 may include a high pressure and temperature treatment to form the treated cultivated plant biomass fractionation 42. The pressure can be generated and controlled by heating in a sealed vessel. The pressure typically ranges from about 100 to about 800 psi. The temperature can range from about 150° C. to about 300° C. (about 300° F. to about 572° F.), with about 200° C. to about 250° C. (about 392° F. to about 482° F.) typically used. The high pressure and temperature treatment can be conducted in a solvent, generally under alkaline conditions. Often, an ethanol and water mixture may be used as the solvent. Other alcohols or water mixtures may also be used in component separation processing 22. This high pressure and temperature treatment may serve to breakdown and solubilize the hemicellulose and lignin components of cultivated plant biomass. Because both the hemicellulose and lignin are solubilized, the lignin and hemicellulose mixture 28 can be later separated from the insoluble cellulose 26. Also, extractables may be removed and recovered/recycled from this treatment as well as any chemicals like alcohols.

After the high pressure and temperature treatment, the treated cultivated plant biomass fractionation 42 may then be attained. Alternatively, the cultivated plant biomass fractionation 16 or filtered cultivated plant biomass fractionation 20 can also be broken down with other processes in the component separation processing 22 that may include at least one of kraft processing, sulfite pulping, pyrolysis, steam explosion, ammonia fiber explosion, dilute acid hydrolysis, alkaline hydrolysis, alkaline oxidative treatment, and enzymatic treatment. No matter what process is used within the component separation processing 22, the cultivated plant biomass can be broken down to the treated cultivated plant biomass fractionation 42 after the component separation processing 22 is completed. During the component separation processing 22, the hemicellulose component may hydrolyze the easiest whereas cellulose may be the most difficult to hydrolyze. This hydrolyzation can help to separate the component streams of the cultivated plant biomass. From this hydrolysis, a physical division of the component streams may occur.

From there, a second filtration 24 can be done on the treated cultivated plant biomass fractionation 42 in order to separate the cellulose 26 from the lignin and hemicellulose mixture 28. This second filtration 24 serves to remove the insoluble cellulose from the soluble lignin and hemicellulose mixture 28. Optionally, the insoluble cellulose can be washed with water or a chemical like aqueous ethanol and separated from the wash in the second filtration 24. The filtration leaves an aqueous mixture of hemicellulose sugars and solubilized lignin. The residual chemical(s) can be removed from this filtrate through concentration or distillation by applying a low to modest temperature and a minimal vacuum which may be sufficient to evaporate the chemical in the residual chemical removal 30 of FIG. 1. When the chemical is ethanol, this temperature may be about 25° C. to about 40° C. (about 77° F. to about 104° F.) and the pressure typically may vary from about 30 to about 70 millimeters of mercury. The chemical may then be recycled for reuse. Ideally, 100% of the chemical would be recovered so that it can be recycled back into the process, which reduces costs associated with purchasing additional chemicals. Typically, at least 90% may be recovered for recycling. This second filtration 24 also can assist in separating the solubilized lignin and hemicellulose mixture 28 from the insoluble and solid cellulose 26. After this step, the separated cellulose 26 can undergo the production of biobased chemicals 40.

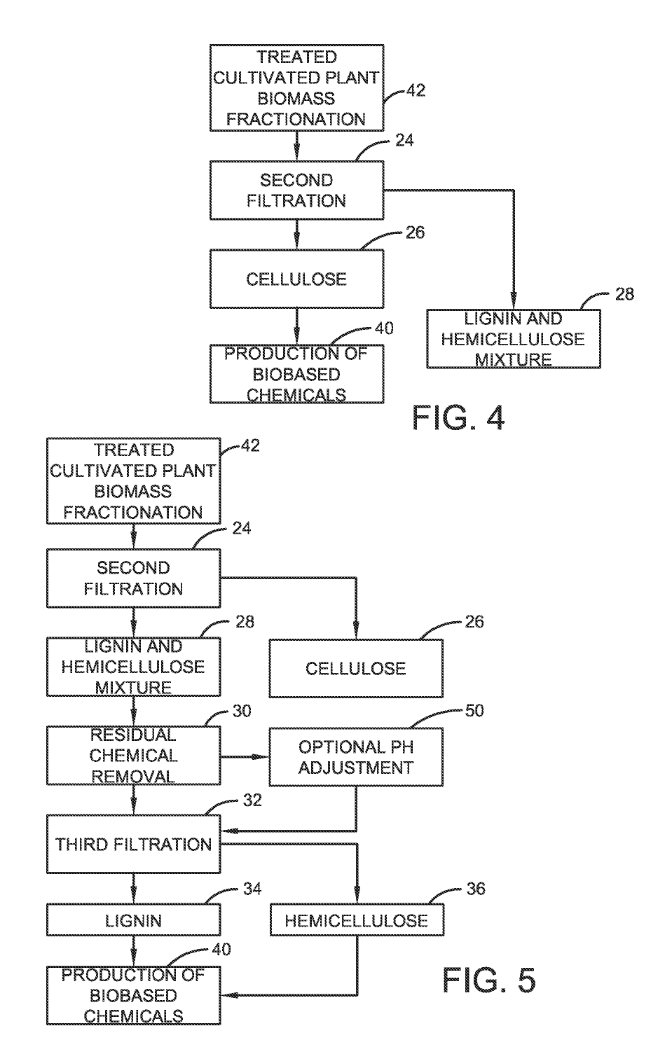

FIG. 4 is a flow diagram schematically depicting the process in which the treated cultivated plant biomass fractionation 42 can provide cellulose 26, which may be further processed to produce derivative products in accordance with an embodiment of the present invention. In separating the cellulose 26 after the second filtration 24, the cellulose 26 can then be processed to allow for the production of biobased chemicals 40. The second filtration 24 also may provide a way to obtain the soluble lignin and hemicellulose mixture 28. For instance, the cellulose 26 can be hydrolyzed, reacted, and purified to provide for the production of biobased chemicals 40, namely cellulosic esters, aliphatic carboxylic acids, aliphatic esters, polyols, furans, dihydrofuran, tetrahydrofurans, lactones, and ethanol. Some of these biobased chemicals from cellulose 26 can include but are not limited to cellulose acetate, cellulose propionate, cellulose benzoate, methyl and ethyl adipate, methyl and ethyl levulinate, methyl and ethyl succinate, methyl and ethyl 2,5-furandicarboxylate, adipic acid, levulinic acid, succinic acid, 2,5-furandicarboxylic acid, 3,4-dehydro-γ-valerolactone, γ-valerolactone, 2-methyltetrahydrofuran, sorbitol, hexane-1,6-diol, pentane-1,4-diol, butane-1,4-diol, 2,5-di(hydroxymethyl)furan, 2,5-di(hydroxymethyl)tetrahydrofuran, glyercol, propylene glycol, and ethanol.

FIG. 5 is a flow diagram schematically depicting the treated cultivated plant biomass fractionation 42 which can be further processed to obtain lignin 34 and hemicellulose 36 in accordance with an embodiment of the present invention. After the treated cultivated plant biomass fractionation 42 is subjected to a second filtration 24, a lignin and hemicellulose mixture 28 may be attained. From this step, a residual chemical removal 30 can then be completed. In addition to chemicals added during the component separation processing 22 shown in FIG. 3, the residual chemical removal 30 can remove any chemicals carried over from the optional chemical processing 14 shown in FIG. 2, which may also be recycled back into the process. In the residual chemical removal 30, the chemical, typically an alcohol like ethanol can be recovered through concentration or distillation by applying a low to modest temperature and a minimal vacuum which may be sufficient to evaporate the chemical in the residual chemical removal 30. When the chemical is ethanol, this temperature may be about 25° C. to about 40° C. (about 77° F. to about 104° F.) and the pressure typically may vary from about 30 to about 70 millimeters of mercury. The chemical may then be recovered and recycled for reuse. After a third filtration 32, the mixture can then be separated into lignin 34 and hemicellulose 36. In some instances, the processing may require an optional pH adjustment 50 using an acid to adjust the pH of the solution to a point which the lignin and hemicellulose can be efficiently separated from each other prior to the third filtration 32. Typically, sulfuric acid can be used in the optional pH adjustment 50, but other acids may be employed. Optionally, the precipitated lignin can be washed with water and separated from the wash in the third filtration 32. In the third filtration 32, the hemicellulose 36 can be primarily soluble and may be in an aqueous solution of the filtrate. The optional removal of the water from the hemicellulose 36 provides a concentrated form of hemicellulose sugars. The separation of the component streams to lignin 34 and hemicellulose 36 can permit the production of biobased chemicals 40. Lignin 34 can be a source of aromatic chemicals like aromatic carboxylic acids, aromatic esters, aromatic aldehydes, aryl alcohols, aryl ketones, styrenes, aryl ethanes, aryl propenes, aryl propanes, cresols, phenols, benzenes, and pyrolytic oils. Some of the specific biobased chemicals from lignin 34 can include but are not limited to methyl and ethyl 4-hydroxybenzoate, methyl and ethyl vanillate, methyl and ethyl syringate, 4-hydroxybenzoic acid, (4-hydroxyphenyl)acetic acid, vanillic acid, homovanillic acid, syringic acid, homosyringic acid, 4-hydroxybenzaldehyde, vanillin, syringaldehyde, 4-hydroxybenzyl alcohol, 2-(4-hydroxyphenyl)ethanol, vanillyl alcohol, homovanillyl alcohol, syringyl alcohol, homosyringyl alcohol, 4-hydroxyacetophenone, acetoguaiacone, acetosyringone, 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, iso-syringeugenol, ethyl phenol, ethyl guaiacol, ethyl syringol, propyl phenol, propyl guaiacol, propyl syringol, cresol, creosol, syringyl creosol, phenol, guaiacol, syringol, benzene, toluene, xylene, ethyl benzene, propyl benzene, biphenyl, and pyrolytic oils. Hemicellulose 36 can provide furans, dihydrofurans, tetrahydrofurans, polyols, lactones, and butenes. Some of the specific biobased chemicals from hemicellulose 36 may include but are not limited to furfural, γ-butyrolactone, tetrahydrofuran, ribitol, xylitol, arabitol, glyercol, propylene glycol, and isoprene.

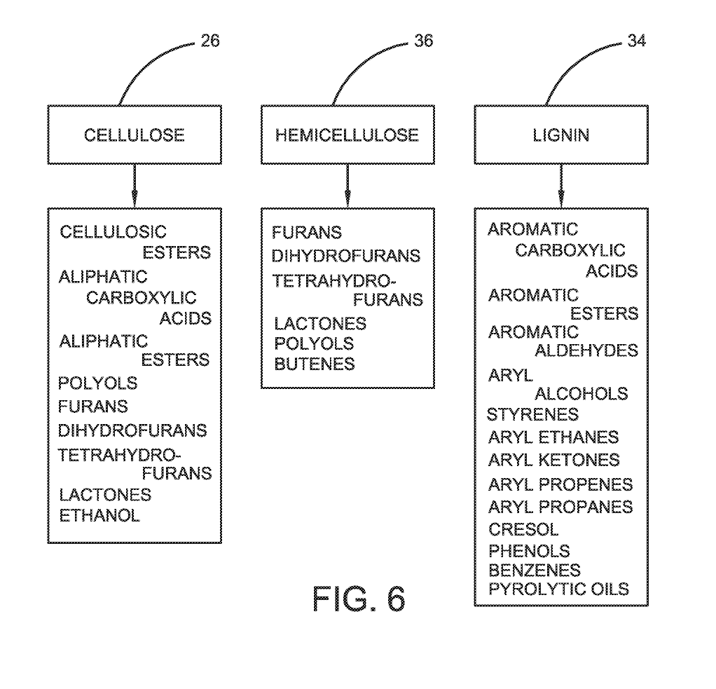

FIG. 6 is a flow diagram schematically depicting the plurality of the component streams and their conversion to derivative biobased products in accordance with an embodiment of the present invention. It shows the production of some derivative products from the plurality of component streams, namely cellulose 26, lignin 34, and hemicellulose 36. The processes described herein may provide only one independent and separate component stream or a plurality of component streams. These derivative biobased product(s) may be obtained from only one independent and separate component stream or more that one of the component streams. Each component stream may provide only one derivative product or more than one derivative product, which may also be used in the production of another chemical or other chemicals. A derivative product or a plurality of derivative products may be commodity, fine, and/or specialty chemicals, and be produced through at least one of chemical processing, biological processing, catalytic processing, and/or pyrolytic processing. These products can be at least one of aromatic chemicals, aliphatic chemicals, heterocyclic chemicals, and fuels. These products can be at least one of aromatic carboxylic acids, aromatic esters, aromatic aldehydes, aryl alcohols, aryl ketones, styrenes, aryl ethanes, aryl propenes, aryl propanes, cresols, phenols, benzenes, pyrolytic oils, cellulosic esters, aliphatic carboxylic acids, aliphatic esters, polyols, ethanol, furans, dihydrofuran, tetrahydrofurans, lactones, ethanol, and butenes. For example, aliphatic carboxylic acids may include but are not limited to adipic acid, levulinic acid and succinic acid. For instance, polyols may include but are not limited to sorbitol, xylitol, arabinitol, hexane-1,6-diol, pentane-1,4-diol, butane-1,4-diol, 2,5-hydroxymethylfuran, 2,5-hydroxymethyltetrahydrofuran, glyercol, propylene glycol. For example, aromatic aldehydes may include but are not limited to 4-hydroxybenzaldehyde, vanillin, and syringealdehyde. For instance, benzenes may include benzene, toluene, xylene, and biphenyl. Since the process can generate a plurality of component streams which may then be used for the production of biobased chemicals, waste can be minimized. The residual cultivated plant biomass waste from this process can be less than 25%. It can also be less than 15%. The waste from the process may also be used to produce energy, including heat and/or power. This method for reducing waste can provide a greener process where the majority of the cultivated plant biomass provided at the beginning of the process can be converted into usable products in the production of biobased chemicals.

FIG. 7 is a flow diagram schematically depicting an illustrative flow of the cultivated plant biomass treatment and processing along with the recovery of chemicals 44 used within the process in accordance with an embodiment of the present invention. In this diagram, the chemicals used for treating the cultivated plant biomass 10 in the optional chemical processing 14, the first filtration 18, the component separation processing 22, and the second filtration 24 can be recoverable and recyclable for reuse. First, the cultivated plant biomass 10 may undergo a mechanical processing 12. After the optional chemical processing 14, the cultivated plant biomass fractionation 16 can be formed. Then, a first filtration 18 may be performed. Typically, the chemical for the optional chemical processing 14 is an alcohol like ethanol. After the first filtration 18, there may be a recovery of chemicals 44 in which the chemical can be removed from the filtered cultivated plant biomass fractionation 20. Besides the recovery of chemicals 44 from the first filtration 18, an analogous recovery of chemicals 44 may be applicable from the residual chemical removal 30. From the recovery of chemicals 44, the chemical may be subjected to a distillation and/or filtration 46, and can then be placed into a chemical holding tank 48 for reuse in one or more of the optional chemical processing 14, the washes of the first filtration 18, the component separation processing 22, or the washes of the second filtration 24 steps. Ideally, 100% of the chemicals used in the process would be recovered. Preferably, at least a 90% recovery can provide a greener process where fewer chemicals are used and costs associated with purchasing more chemicals from the recovery loss are minimized. Additionally, during this process, the recovery of chemicals from the component stream can be processed to derivative products.

The flow diagrams depicted herein are provided merely as an example to clearly and concisely describe embodiments of a method within the scope of the present invention. Some steps may be skipped or modified, new steps may be added, existing steps may be deleted, or the order of steps may be altered from that shown in the flow diagram without departing from the scope of the present invention. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

I/We claim:

1. A method for biorefining, comprising the steps of:
providing cultivated plant biomass;
processing said cultivated plant biomass to provide a plurality of component streams; and
producing derivative products from said plurality of component streams.

2. The method of claim 1, wherein said cultivated plant biomass comprises cultivated plants, cultivated trees, cultivated shrubs, and cultivated bushes.

3. The method of claim 1, wherein said cultivated plant biomass comprises at least one of aquatic plants, medicinal plants, fiber plants, ornamental plants, and grassy plants.

4. The method of claim 1, wherein said cultivated plant biomass comprises at least one of annual cultivated plants, native annual cultivated plants, hybrid annual cultivated plants, and genetically modified annual cultivated plants.

5. The method of claim 1, wherein said cultivated plant biomass comprises at least one of perennial cultivated plants, native perennial cultivated plants, hybrid perennial cultivated plants, and genetically modified perennial cultivated plants.

6. The method of claim 1, wherein said cultivated plant biomass comprises at least one of biennial cultivated plants, native biennial cultivated plants, hybrid biennial cultivated plants, and genetically modified biennial cultivated plants.

7. The method of claim 1, wherein said cultivated plant biomass comprises cultivated plant residues of at least one of cultivated plants, cultivated trees, cultivated shrubs, cultivated bushes, aquatic plants, medicinal plants, fiber plants, ornamental plants, grassy plants, annual cultivated plants, native annual cultivated plants, hybrid annual cultivated plants, genetically modified annual cultivated plants, perennial cultivated plants, native perennial cultivated plants, hybrid perennial cultivated plants, genetically modified perennial cultivated plants, biennial cultivated plants, native biennial cultivated plants, hybrid biennial cultivated plants, and genetically modified biennial cultivated plants.

8. The method of claim 1, wherein said plurality of component streams comprises lignin, cellulose, and hemicellulose.

9. The method of claim 1, wherein said step of processing said cultivated plant biomass to provide a plurality of component streams comprises mechanical processing and component separation processing.

10. The method of claim 9, wherein said mechanical processing comprises at least one of chopping, chipping, cutting, shredding, debarking, milling, and grinding.

11. The method of claim 9, wherein said step of component separation processing, further comprises the step of:
providing cellulose.

12. The method of claim 9, wherein components resulting from said component separation processing comprise lignin and hemicellulose.

13. The method of claim 9, wherein the step of processing said cultivated plant biomass to provide a plurality of component streams, further comprises the step of:
completing a chemical processing during said processing of said cultivated plant biomass.

14. The method of claim 13, wherein said chemical processing comprises at least one of solvent treatment, acidic treatment, basic treatment, and enzymatic treatment.

15. The method of claim 13, further comprising the step of:
removing extractables from said chemical processing.

16. The method of claim 15, wherein said step of removing extractables from said chemical processing further comprises the step of at least one of:
extracting metals from ore, lubricating, cleaning, disinfecting, deodorizing, scenting, and producing biofuels.

17. The method of claim 13, further comprising the steps of:
recovering chemicals from said chemical processing; and
recycling said chemicals from said chemical processing.

18. The method of claim 9, further comprising the step of:
using a residual chemical removal in said processing of said cultivated plant biomass.

19. The method of claim 18, wherein said step of using residual chemical removal produces hemicellulose and lignin.

20. The method of claim 18, wherein said step of using residual chemical removal further comprises the steps of:
adjusting a pH; and
producing hemicellulose and lignin.

21. The method of claim 18, further comprising the steps of:
recovering at least on chemical from said residual chemical removal; and
recycling said at least one chemical from said residual chemical removal.

22. The method of claim 9, wherein the step of processing said cultivated plant biomass to provide said plurality of component streams further comprising the step of:
utilizing an additional treatment during processing of said cultivated plant biomass.

23. The method of claim 22, wherein said additional treatment comprises at least one of heat treatment, pressure treatment, kraft pulping, sulfite pulping, pyrolysis, steam explosion, ammonia fiber explosion, dilute acid hydrolysis, alkaline hydrolysis, alkaline oxidative treatment, and enzymatic treatment.

24. The method of claim 23, further comprising the step of selectively utilizing at least two of said additional treatments for said processing said cultivated plant biomass.

25. The method of claim 23, further comprising the step of removing extractables from said additional treatment.

26. The method of claim 23, further comprising the steps of:
recovering at least one chemical from said additional treatment; and
recycling said at least one chemical from said additional treatment.

27. The method of claim 1, further comprising the step of selectively utilizing one of said component streams for producing said derivative products.

28. The method of claim 1, further comprising the step of selectively utilizing at least two of said component streams for producing said derivative products.

29. The method of claim 1, wherein said plurality of component streams is a mixture of said plurality of component streams.

30. The method of claim 1, wherein at least one component stream of said plurality of component streams is an independent and separate component stream from said plurality of component streams.

31. The method of claim 30, further comprising the step of selectively producing one derivative product from said independent and separate component stream.

32. The method of claim 30, further comprising the step of selectively producing at least two derivative products from said independent and separate component stream.

33. The method of claim 1, further comprising the step of producing at least one derivative product from a residue component stream.

34. The method of claim 1, wherein said producing derivative products comprises at least one of commodity chemicals, fine chemicals, and specialty chemicals.

35. The method of claim 1, wherein said producing derivative products comprises at least one of chemical processing, biological processing, catalytic processing, and pyrolytic processing.

36. The method of claim 1, wherein one of said component streams is lignin, wherein derivative products from lignin comprise at least one of aromatic chemicals and fuels.

37. The method of claim 36, wherein said derivative products comprise at least one of aromatic carboxylic acids, aromatic esters, aromatic aldehydes, aryl alcohols, aryl ketones, styrenes, aryl ethanes, aryl propenes, aryl propanes, cresols, phenols, benzenes, and pyrolytic oils.

38. The method of claim 36, wherein said derivative products comprise at least one of methyl and ethyl 4-hydroxybenzoate, methyl and ethyl vanillate, methyl and ethyl syringate, 4-hydroxybenzoic acid, (4-hydroxyphenyl)acetic acid, vanillic acid, homovanillic acid, syringic acid, homosyringic acid, 4-hydroxybenzaldehyde, vanillin, syringaldehyde, 4-hydroxybenzyl alcohol, 2-(4-hydroxyphenyl)ethanol, vanillyl alcohol, homovanillyl alcohol, syringyl alcohol, homosyringyl alcohol, 4-hydroxyacetophenone, acetoguaiacone, acetosyringone, 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, iso-syringeugenol, ethyl phenol, ethyl guaiacol, ethyl syringol, propyl phenol, propyl guaiacol, propyl syringol, cresol, creosol, syringyl creosol, phenol, guaiacol, syringol, benzene, toluene, xylene, ethyl benzene, propyl benzene, biphenyl, and pyrolytic oils.

39. The method of claim 1, wherein one of said components streams is cellulose, wherein derivative products from cellulose comprise at least one of aliphatic chemicals, heterocyclic chemicals, and fuels.

40. The method of claim 39, wherein said derivative products comprise at least one of cellulosic esters, aliphatic carboxylic acids, aliphatic esters, polyols, furans, dihydrofurans, tetrahydrofurans, lactones, and ethanol.

41. The method of claim 39, wherein said derivative products comprise at least one of cellulose acetate, cellulose propionate, cellulose benzoate, methyl and ethyl adipate, methyl and ethyl levulinate, methyl and ethyl succinate, methyl and ethyl 2,5-furandicarboxylate, adipic acid, levulinic acid, succinic acid, 2,5-furandicarboxylic acid, 3,4-dehydro-γ-valerolactone, γ-valerolactone, 2-methyltetrahydrofuran, sorbitol, hexane-1,6-diol, pentane-1,4-diol, butane-1,4-diol, 2,5-di(hydroxymethyl)furan, 2,5-di(hydroxymethyl)tetrahydrofuran, glyercol, propylene glycol, and ethanol.

42. The method of claim 1, wherein one of said component streams is hemicellulose, wherein said derivative products from hemicellulose comprise at least one of aliphatic chemicals, heterocyclic chemicals, and fuels.

43. The method of claim 42, wherein said derivative products comprise at least one of polyols, furans, dihydrofurans, tetrahydrofurans, lactones, and butenes.

44. The method of claim 42, wherein said derivative products comprise at least one of furfural, γ-butyrolactone, tetrahydrofuran, ribitol, arabitol, xylitol, glyercol, propylene glycol, and isoprene.

45. The method of claim 1, wherein at least one of said plurality of derivative products comprises achiral, racemic, and optically pure products.

46. The method of claim 1, further comprising the step of:
using said at least one derivative product in the production of other chemicals, materials, and products.

47. The method of claim 1, wherein said cultivated plant biomass has a weight, and a waste product of said cultivated plant biomass is less than 25% of said cultivated plant biomass weight.

48. The method of claim 1, wherein said cultivated plant biomass has a weight, and a waste product of said cultivated plant biomass is less than 15% of said cultivated plant biomass weight.

49. The method of claim 47, further comprising the step of:
producing energy utilizing said waste product.

50. A method for biorefining, comprising the steps of:
providing cultivated plant biomass;
processing said cultivated plant biomass to provide a plurality of component streams resulting in at least one waste product; and
utilizing said at least one waste product to produce energy.

51. The method of claim 50, wherein said energy is heat or power.

52. A method for biorefining, comprising the steps of:
providing cultivated plant biomass comprising at least one of cultivated plants, cultivated trees, cultivated shrubs, cultivated bushes, aquatic plants, medicinal plants, fiber plants, ornamental plants, and grassy plants, annual cultivated plants, native annual cultivated plants, hybrid annual cultivated plants, genetically modified annual cultivated plants, perennial cultivated plants, native perennial cultivated plants, hybrid perennial cultivated plants, genetically modified perennial cultivated plants, biennial cultivated plants, native biennial cultivated plants, hybrid biennial cultivated plants, and genetically modified biennial cultivated plants;
providing said cultivated plant biomass comprising cultivated plant residues of at least one of cultivated plants, cultivated trees, cultivated shrubs, cultivated bushes, aquatic plants, medicinal plants, fiber plants, ornamental plants, grassy plants, annual cultivated plants, native annual cultivated plants, hybrid annual cultivated plants, genetically modified annual cultivated plants, perennial cultivated plants, native perennial cultivated plants, hybrid perennial cultivated plants, genetically modified perennial cultivated plants, biennial cultivated plants, native biennial cultivated plants, hybrid biennial cultivated plants, and genetically modified biennial cultivated plants;
processing said cultivated plant biomass comprising of mechanical processing, component separation processing, optional chemical processing, residual chemical removal, and an additional treatment;
providing a plurality of component streams comprising lignin, cellulose, and hemicellulose from said cultivated plant biomass;
recovering chemicals used in said chemical processing and said residual chemical removal for recycling;
removing extractables from said optional chemical processing and said additional treatment;
reducing the waste product of the cultivated plant biomass, wherein said cultivated plant biomass has a weight, and said waste product of said cultivated plant biomass is less than 25% of said cultivated plant biomass weight;
producing energy utilizing said waste product;
producing at least one of commodity chemicals, fine chemicals, and specialty chemicals; and
producing at least one of aromatic carboxylic acids, aromatic esters, aromatic aldehydes, aryl alcohols, aryl ketones, styrenes, aryl ethanes, aryl propenes, aryl propanes, cresols, phenols, benzenes, pyrolytic oils, cellulosic esters, aliphatic carboxylic acids, aliphatic esters, polyols, ethanol, furans, dihydrofurans, tetrahydrofurans, lactones, and butenes from at least one of said component streams.
